(19) United States
(12) Patent Application Publication
(10) Pub. No.: US 2004/0229933 A1
(43) Pub. Date: Nov. 18, 2004

(54) Treatment of hypertension

(75) Inventor: Sidath Dhammika Katugampola, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/782,021

(22) Filed: Jul. 16, 2004

Related U.S. Application Data
(60) Provisional application No. 60/454,052, filed on Mar. 12, 2003.

(30) Foreign Application Priority Data
Feb. 21, 2003 (GB) 0304021.9

(51) Int. Cl. A61K 31/535; A61K 31/40

(52) U.S. Cl. 514/423

(57) ABSTRACT

The present invention relates to the use of a modulator of the Kiss-1 receptor for the treatment of blood pressure disorders, in particular to the use of a Kiss-1 receptor antagonist for the treatment of hypertension. The invention also provides methods of screening for compounds useful in the treatment of hypertension.

This application claims priority, under 35 U.S.C. §119(a), from United Kingdom Patent Application number 0304021.9, which was filed Feb. 21, 2003, and under 35 U.S.C. §119(e) from U.S. patent application Ser. No. 60/454052 which was filed Mar. 12, 2003, the disclosure of which are hereby incorporated by reference.

### FIELD OF THE INVENTION

The present invention relates to the use of a modulator of the Kiss-1 receptor (GPR54) for the treatment of blood pressure disorders, preferably the use of a Kiss-1 receptor antagonist for the treatment of hypertension.

The present invention also relates to a method of treatment of hypertension.

The present invention also relates to assays to screen for compounds useful in the treatment of hypertension.

### BACKGROUND

Blood pressure (BP) is defined by a number of haemodynamic parameters taken either in isolation or in combination. Systolic blood pressure (SBP) is the peak arterial pressure attained as the heart contracts. Diastolic blood pressure (DBP) is the minimum arterial pressure attained as the heart relaxes. The difference between the SBP and the DBP is defined as the pulse pressure (PP).

Hypertension, or elevated BP, has been defined as a SBP of at least 140 mmHg and/or a DBP of at least 90 mmHg. By this definition, the prevalence of hypertension in developed countries is about 20% of the adult population, rising to about 60-70% of those aged 60 or more, although a significant fraction of these hypertensive subjects have normal BP when this is measured in a non-clinical setting. Some 60% of this older hypertensive population have isolated systolic hypertension (ISH), i.e. they have an elevated SBP and a normal DBP. Hypertension is associated with an increased risk of stroke, myocardial infarction, atrial fibrillation, heart failure, peripheral vascular disease and renal impairment (Fagard, RH; (2002) Am. J. Geriatric Cardiology 11(1), 23-28; Brown, M J and Haycock, S; (2000) Drugs 59(Suppl 2), 1-12).

The pathophysiology of hypertension is the subject of continuing debate. While it is generally agreed that hypertension is the result of an imbalance between cardiac output and peripheral vascular resistance, and that most hypertensive subjects have normal cardiac output and increased peripheral resistance there is uncertainty which parameter changes first (Beevers, G et al.; (2001) BMJ 322, 912-916).

Despite the large number of drugs available in various pharmacological categories, including diuretics, alpha-adrenergic antagonists, beta-adrenergic antagonists, calcium channel blockers, angiotensin converting enzyme (ACE) inhibitors and angiotensin receptor antagonists, the need for an effective treatment of hypertension is still not satisfied.

Kiss-1 is a human metastasis suppressor gene that suppresses metastases of human melanomas (Lee, J. H. et al (1996) J. Natl. Cancer Inst. 88, 1731-1737; Erratum in (1997) J. Natl. Cancer Inst. 89, 1549). It shows all characteristics of a secreted neuropeptide, with a putative signal sequence, several possible cleavage sites, including an amidation/cleavage site that would result in a number of amidated peptide fragments of various lengths. Ohtaki et al ((2001) Nature 411, 613-617) identified a carboxy-terminally amidated peptide with 54 amino acid residues (Kiss-1 (1-54)), which has also been called metastin, isolated from extracts from human placenta. Kiss-1 was found to inhibit tumour metastasis and play a role in endocrine functions (Ohtaki et al. (2001) Nature 411, 613-617 and Kotani et al. (2001) J. Biol Chem. 276, 34631-34636).

GPR54 was originally identified as an orphan G-protein coupled receptor with some sequence similarity to receptors for the neuropeptide galanin. It was then found that Kiss-1, as well as C-terminal fragments of Kiss-1 such as Kiss-1 (40-54) and Kiss-1 (45-54), are potent agonists for this receptor (Muir, I. A. et al (2001) J. Biol. Chem. 276, 28969-28975; Ohtaki, T. et al (2001) Nature 411, 613-617; Kotani et al. (2001) J. Biol Chem. 276, 34631-34636). It has been shown that Kiss-1 inhibits migration, chemotaxis and invasion of CHO cells transfected with GPR54.

Surprisingly, we have found that Kiss-1 also has vascular effects. In an organ bath system using rat aortic rings, we found that Kiss-1 is a potent vasoconstrictor. Kiss-1 agonists are therefore likely to increase blood pressure and therefore have utility in treating hypotension, whereas Kiss-1 antagonists will decrease blood pressure and therefore be useful in the treatment of hypertension.

### BRIEF DESCRIPTION OF THE DRAWING

FIG. 1 is a dose response curve for vasoconstriction by Kiss-1 (45-54) in rat aortic rings in an organ bath system.

### DETAILED DESCRIPTION OF THE INVENTION

A seminal finding of the present invention is the ability to treat hypertension with an antagonist for the Kiss-1 receptor, and/or to treat hypotension with an agonist for the Kiss-1 receptor.

Therefore the invention relates to Kiss-1 receptor antagonists for use in the treatment of hypertension. The invention also relates to the use of Kiss-1 receptor antagonists for the manufacture of a medicament for the treatment of hypertension. The invention also relates to a method of treatment of hypertension with an antagonist to the Kiss-1 receptor. One aspect of the invention is therefore a method of treating hypertension, comprising the administration to a patient in need of such treatment of an effective amount of a Kiss-1 receptor antagonist. The term “hypertension” includes all diseases characterised by supranormal blood pressure, such as essential hypertension, pulmonary hypertension, secondary hypertension, isolated systolic hypertension, hypertension associated with diabetes, hypertension associated with atherosclerosis, and renovascular hypertension. The term “treating hypertension” includes the palliative, curative and prophylactic treatment of hypertension, complications arising from hypertension, and other associated co-morbidities, including congestive heart failure, angina, stroke and the like.

The Kiss-1 receptor antagonists will preferably have an IC₅₀ in a ligand binding assay of less than 100 nM, more preferably an IC₅₀ of less than 10 nM, even more preferably an IC₅₀ of less than 1 nM. The IC₅₀ may be measured in a ligand binding assay, e.g. as described in Example 1, or by measuring the inhibition of agonist-induced second messenger responses (see, for example, Example 2), or a pA₂ can be measured in an isolated vascular tissue preparation (see, for example, Example 3).

Preferably the Kiss-1 receptor antagonists will be at least 10 fold selective over galanin receptor type 2, more preferably at least 100 fold selective over galanin receptor type 2, even more preferably at least 1000 fold selective over galanin receptor type 2. Preferably the Kiss-1 receptor antagonists will be at least 10 fold selective over galanin receptor type 3, more preferably at least 100 fold selective over galanin receptor type 3, even more preferably at least 1000 fold selective over galanin receptor type 3.

Suitable Kiss-1 receptor agonists include Kiss (45-54), derived from the 54 amino acid Kiss peptide sequence.

Suitable antagonists can be antibodies to the Kiss-1 receptor, modified Kiss-derived peptides which retain their binding affinity, but are unable to activate the receptor, or small molecules which can be identified by screening compounds or compound libraries with, for example, a ligand binding assay as described in Example 1. Other assay formats can also be used, e.g. fluorescence-based assays (e.g. a FLIPR-based assay as described in Example 2) or reporter-gene based assays, measuring the inhibition of the activation of the receptor by Kiss peptide or an alternative suitable agonist by test compounds.

Yet a further aspect of the invention is a method of screening for compounds useful for treating hypertension, comprising screening compounds for antagonist activity against Kiss-1 receptor, and selecting compounds with an IC₅₀ of less than 100 nM, preferably with an IC₅₀ of less than 10 nM, even more preferably with an IC₅₀ of less than 1 nM. Another aspect of the invention is the use of a compound identified by this method in the manufacture of a medicament for the treatment of hypertension.

Another aspect of the invention is a process for providing a medicament for the treatment of hypertension, comprising the following steps:

(a) testing compounds in a ligand binding assay against the Kiss-1 receptor;

(b) selecting a compound with an IC₅₀ of less than 100 nM;

(c) formulating a compound with the same structure as that selected in step (b), or a pharmaceutically acceptable salt thereof, with a pharmaceutically acceptable carrier or excipient; the process may also comprise the additional steps of:

(d) packaging the formulation of step (c); and

(e) making the package of step (d) available to a patient suffering from hypertension.

Preferably, the compound selected in step (b) will have an IC₅₀ of less than 10 nM, even more preferably it will have an IC₅₀ of less than 1 nM.

Yet another aspect of the invention is a process for providing a medicament for the treatment of hypertension, comprising the following steps:

(a) testing compounds in an assay, measuring the inhibition of the agonist-stimulated second messenger response of Kiss-1 receptors;

(b) selecting a compound with an IC₅₀ of less than 100 nM;

(c) formulating a compound with the same structure as that selected in step (b), or a pharmaceutically acceptable salt thereof, with a pharmaceutically acceptable carrier or excipient; the process may also comprise the additional steps of:

(d) packaging the formulation of step (c); and

(e) making the package of step (d) available to a patient suffering from hypertension.

Preferably, the assay in step (a) measures a transient rise in intracellular calcium in Kiss-1 receptor expressing cells in response to Kiss-1 or another agonist for the Kiss-1 receptor, even more preferably, the transient rise in intracellular calcium is measured by fluorescence techniques, using calcium-sensitive fluorescent dyes such as Fluo-3. Preferably, the compound selected in step (b) will have an IC50 of less than 10 nM, even more preferably it will have an IC₅₀ of less than 1 nM.

Another aspect of the invention is a process for preparing a medicament for the treatment of hypertension, comprising the steps of (a) testing compounds in a ligand binding assay against Kiss-1 receptor or testing compounds in an assay, measuring the inhibition of the agonist-stimulated second messenger response of Kiss-1 receptors, (b) identifying one or more compounds capable of antagonising the Kiss-1 receptor with an IC₅₀ of less than 100 nM; and (c) preparing a quantity of those one or more identified compounds.

Another aspect of the invention is a method of preparing a composition for treating hypertension which comprises:

(a) identifying a compound which specifically binds to the Kiss-1 receptor by a method which comprises contacting cells expressing Kiss-1 receptor or membranes prepared from such cells with a radiolabelled Kiss-1 receptor ligand in the presence or absence of a test compound, measuring the radioactivity bound to the cells or membranes, comparing the radioactivity bound to the cells or membranes in the presence and absence of test compound, whereby a compound which causes a reduction in the radioactivity bound is a compound specifically binding to Kiss-1 receptor; and

(b) admixing said compound with a carrier.

Yet another aspect of the invention is a method of preparing a composition for treating hypertension which comprises:

(a) identifying a compound which specifically binds to and inhibits the activation of a Kiss-1 receptor by a method which comprises separately contacting cells expressing Kiss-1 receptor on their surface and producing a second messenger response in response to Kiss-1 or a Kiss-1 agonist, or a membrane preparation of such cells, with both the compound and an agonist of the Kiss-1 receptor, and with only the agonist, under conditions suitable for activation of the Kiss-1 receptor, and measuring the second messenger response in the presence of only the agonist for the Kiss-1 receptor and in the presence of the agonist and the compound, a smaller change in the second messenger response in the presence of both agonist and compound than in the presence of the agonist only indicating that the compound inhibits the activation of the Kiss-1 receptor; and

(b) admixing said compound with a carrier.

The invention relates to the use of a Kiss-1 receptor antagonist for the treatment of hypertension alone, or in combination with one or more other agents such as angiotensin receptor blockers, angiotensin converting enzyme inhibitors, calcium channel blockers, diuretics, or beta blockers.

The Kiss-1 receptor was first cloned as an orphan receptor from rat brain by Lee, D. K. et al ((1999) FEBS Letters 446, 103-107), and named GPR54. The mouse and human orthologues have also been identified (Clements et al (2001) Biochem. Biophys. Res. Comms. 284, 1189-1193). In one aspect of the present invention, a Kiss-1 receptor may be used as a target in screens to identify compounds capable of modulating Kiss-1 receptors. In this regard, the target may comprise an amino acid sequence as shown in Clements et al ((2001) Biochem. Biophys. Res. Comms. 284, 1189-1193) or a variant, homologue, derivative or fragment thereof which is prepared by recombinant and/or synthetic means or an expression entity comprising same.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

In addition to the use of specific amino acid sequences mentioned herein, the present invention also encompasses the use of variants, homologues and derivatives thereof.

In the present context, a homologous sequence is taken to include an amino acid sequence which may be at least 75, 85 or 90% identical to the amino acid sequence of the human GPR54 shown in Clements et al ((2001) Biochem. Biophys. Res. Comms. 284, 1189-1193), preferably at least 95 or 98% identical. In particular, homology should typically be considered with respect to those regions of the sequence known to be essential for an activity. Although homology can also be considered in terms of similarity (i.e. amino acid residues having similar chemical properties/functions), in the context of the present invention it is preferred to express homology in terms of sequence identity. Such sequence homology/identity can be easily assessed by publicly or commercially available bioinformatics software, such as Blast2 (Altschul, S. F. et al (1997) Nucl. Acids Res. 25, 3389-3402), or programs included in the GCG software package (Devereux et al (1984) Nucl. Acids Res. 12, 387; Wisconsin Package Version 10, Genetics Computer Group (GCG, Madison, Wis.), such as Bestfit or Gap. In most cases, the default parameters offered by the software, e.g. Bestfit or Gap, for Gap Penalties etc. are suitable for this assessment.

Reference to an antagonist, an agonist or an inhibitor shall at all times be understood to include all active forms of such agents, including the free form thereof (e.g. the free and/or base form) and also all pharmaceutically acceptable salts, polymorphs, hydrates, silicates, stereo-isomers (e.g. diastereoisomers and enantiomers) and so forth. Active metabolites of any of the compounds, in any form, are also included.

Particular formulations of the compounds or combination of compounds for oral delivery, intravenous or subcutaneous or intramuscular delivery or for topical application (creams, gels) are included in the invention.

“Potency” as used herein is a measure of the concentration of a compound at which it is effective. The potency of a compound as an antagonist for the receptor can be, for example, determined in a binding assay as described in Example 1, and potency in this context will refer to affinity of the compound for the receptor, measured as the IC₅₀ of the compound, i.e. the concentration inhibiting 50% of the labelled compound from binding to the receptors. The potency of a compound can also be determined in a functional assay such as contractile assays for different tissues expressing different receptor subtypes as described in Example 3. The potency in this case would refer to the IC₅₀ of the compound, i.e. the concentration which inhibits 50% of the functional response seen by application of the agonist.

“Selectivity” as used herein is a measure of the relative potency of a compound between two receptor subtypes for the same endogenous ligand. This can be determined in binding assays as described in Example 1, or in functional assays as described in Examples 2 or 3.

For the avoidance of doubt, the term “compound” may refer to a chemical or biological agent, and includes, for example, antibodies, antibody fragments, other proteins, peptides, sugars, any organic or inorganic molecules. Compounds that may be used for screening include, but are not limited to, peptides such as, for example, soluble peptides, including but not limited to members of random peptide libraries; (see, e.g., Lam et al. (1991) Nature 354, 82-84; Houghten et al. (1991) Nature 354, 84-86), and combinatorial chemistry-derived molecular library made of D- and/or L- configuration amino acids, phosphopeptides (including, but not limited to, members of random or partially degenerate, directed phosphopeptide libraries; see, e.g., Songyang et al. (1993) Cell 72, 767-778), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules.

The skilled person will be well aware how to obtain antibodies or antibody fragments that recognise the Kiss-1 receptor and can then be screened by the methods of the invention for their potential to be suitable for use in the treatment of hypertension or hypotension. For the production of antibodies, various host animals may be immunized by injection with Kiss-1 receptor, a Kiss-1 receptor peptide (e.g. one corresponding to extracellular loops or the extracellular domain), truncated Kiss-1 receptor polypeptides (Kiss-1 receptor in which one or more domains, e.g. the transmembrane domain or cellular domain, has been deleted), functional equivalents of Kiss-1 receptors or mutants of Kiss-1 receptors. Such host animals may include but are not limited to rabbits, mice, hamsters and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of the immunized animals.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, ((1975) Nature 256, 495-497 and U.S. Pat. No. 4,376,110), the human B-cell hybridoma technique (Kosbor et al. (1983) Immunology Today 4, 72; Cole et al. (1983) Proc. Natl. Acad. Sci. USA 80, 2026-2030), and the EBV-hybridoma technique (Cole et al. (1985) Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al. (1984) Proc. Natl. Acad. Sci., 81, 6851-6855; Neuberger et al. (1984) Nature 312, 604-608; Takeda et al. (1985) Nature 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird (1988) Science 242, 423-426; Huston et al. (1988) Proc. Natl. Acad. Sci. USA 85, 5879-5883; and Ward et al. (1989) Nature 334, 544-546) can be adapted to produce single chain antibodies against Kiss-1 receptor gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments or by papain digestion of antibody molecules. Alternatively, Fab expression libraries may be constructed (Huse et al. (1989) Science 246, 1275-1281) to allow rapid and easy identification of monoclonal Fab fragments with the desired specificity.

Antibodies to Kiss-1 receptor may also be obtained by generating anti-idiotype antibodies against the Kiss-1 peptide, using techniques well known to those skilled in the art (see, e.g. Greenspan & Bona (1993) FASEB J 7, 437-444; and Nissinoff (1991) J. Immunol. 147, 2429-2438).

The suitability of the Kiss-1 antagonist can be readily determined by evaluation of their potency and selectivity using methods such as those disclosed herein, followed by evaluation of their toxicity, pharmacokinetics (absorption, metabolism, distribution and elimination), etc in accordance with standard pharmaceutical practice. Suitable compounds are those that are potent and selective, have no significant toxic effect at the therapeutic dose, and preferably are bioavailable following oral administration.

Oral bioavailablity refers to the proportion of an orally administered drug that reaches the systemic circulation. The factors that determine oral bioavailability of a drug are dissolution, membrane permeability and hepatic clearance. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the Kiss-1 antagonists have a minimum solubility of 50 μg/ml. Solubility can be determined by standard procedures known in the art such as described in Lipinski C A et al. (1997) Adv. Drug Deliv. Rev. 23(1-3), 3-25.

Membrane permeability refers to the passage of a compound through the cells of the GIT. Lipophilicity is a key property in predicting this and is determined by in vitro Log D_{7.4} measurements using organic solvents and buffer. Preferably the Kiss-1 antagonists have a Log D_{7.4} of −2 to +4, more preferably −1 to +3. The Log D can be determined by standard procedures known in the art such as described in Stopher, D and McClean, S; (1990) J. Pharm. Pharmacol. 42(2), 144.

Cell monolayer assays such as Caco2 add substantially to prediction of favourable membrane permeability in the presence of efflux transporters such as P-glycoprotein, so-called Caco2 flux. Preferably, the Kiss-1 antagonists have a Caco2 flux of greater than 2×10⁻⁶ cms⁻¹, more preferably greater than 5×10⁻⁶ cms⁻¹. The Caco2 flux value can be determined by standard procedures known in the art such as described in Artursson, P and Magnusson, C; J. Pharm. Sci. (1990) 79(7), 595-600.

Metabolic stability addresses the ability of the GIT to metabolise compounds during the absorption process or the liver to do so immediately post-absorption: the first pass effect. Assay systems such as microsomes, hepatocytes etc are predictive of metabolic lability. Preferably the Kiss-1 antagonists show metabolic stability in the assay system that is commensurate with an hepatic extraction of less then 0.5. Examples of assay systems and data manipulation are described in Obach, R S; (2001) Curr. Opin. Drug Disc. Devel. 4(1), 36-44 and Shibata, Y et al. (2000); Drug Met. Disp. 28(12), 1518-1523.

Because of the interplay of the above processes, further support that a drug will be orally bioavailable in humans can be gained by in vivo experiments in animals. Absolute bioavailability is determined in these studies by administering the compound separately or in mixtures by the oral route. For absolute determinations (% orally bioavailable) the intravenous route is also employed. Examples of the assessment of oral bioavailability in animals can be found in Ward, K W et al. (2001); Drug Met. Disp. 29(1), 82-87; Berman, J et al. (1997); J. Med. Chem. 40(6), 827-829 and Han K S and Lee M G (1999); Drug Met. Disp. 27(2), 221-226.

The compounds of the invention can be administered alone but will generally be administered in admixture with a suitable pharmaceutical excipient, diluent or carrier selected with regard to the intended route of administration and standard pharmaceutical practice.

For example, the compounds of the invention can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The compounds of the invention may also be administered as fast-dispersing or fast-dissolving dosage forms or in the form of a high energy dispersion or as coated particles. Suitable formulations may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

The following formulation examples are illustrative only and are not intended to limit the scope of the invention. Active ingredient means a compound of the invention.

Formulation 1:

A tablet is prepared using the following ingredients:

Active ingredient (50 mg) is blended with cellulose (microcrystalline), silicon dioxide, stearic acid (fumed) and the mixture is compressed to form tablets.

Formulation 2:

An intravenous formulation may be prepared by combining active ingredient (100 mg) with isotonic saline (1000 ml)

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the Kiss-1 antagonists may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intracavernouslly, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

The dosage of the combination of the invention in such formulations will depend on its potency, but can be expected to be in the range of from 1 to 500 mg of Kiss-1 antagonist for administration up to three times a day. A preferred dose is in the range 10 to 100 mg (e.g. 10, 25, 50 and 100 mg) of Kiss-1 antagonist which can be administered once, twice or three times a day (preferably once). However the precise dose will be as determined by the prescribing physician and will depend on the age and weight of the subject and severity of the symptoms.

For oral and parenteral administration to human patients, the daily dosage level of a compound of the invention will usually be from to 5 to 500 mg/kg (in single or divided doses).

Thus tablets or capsules may contain from 5 mg to 250 mg (for example 10 to 100 mg) of the compound of the invention for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will appreciate that the compounds of the invention may be taken as a single dose as needed or desired (i.e. prn). It is to be appreciated that all references herein to treatment include acute treatment (taken as required) and chronic treatment (longer term continuous treatment).

The compounds of the invention can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the compounds of the invention and a suitable powder base such as lactose or starch.

Aerosol or dry powder formulations are preferably arranged so that each metered dose or “puff” contains from 1 μg to 50 mg of a compound of the invention for delivery to the patient. The overall daily dose with an aerosol will be in the range of from 1 μg to 50 mg which may be administered in a single dose or, more usually, in divided doses throughout the day.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch, depot or subcutaneous injection. They may also be administered by the pulmonary or rectal routes.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in published international patent applications WO91/11172, WO94/02518 and WO98/55148.

Oral administration of the compounds of the invention is a preferred route, being the most convenient. In circumstances where the recipient suffers from a swallowing disorder or from impairment of drug absorption after oral administration, the drug may be administered parenterally, sublingually or buccally.

### EXAMPLES

The examples below are carried out using standard techniques, which are well-known and routinely used by those skilled in the art; the examples illustrate but do not limit the invention.

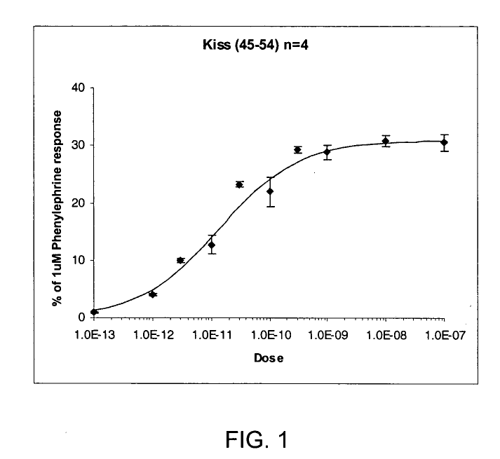

FIG. 1 shows the vasoconstriction by Kiss-1 (45-54) in an organ bath system of rat aortic rings.

### Example 1

### Ligand Binding Assays to Determine Affinity and Selectivity of Kiss-1 Antagonists

The cDNA of the human Kiss-1 receptor coding region was obtained by polymerase chain reaction (PCR), using primers designed around the ATG start codon and the stop codon of the Kiss-1 receptor sequence, which can be found, for example, in patent application WO 02/059344 as SEQ ID No: 1, where it is called GPR54. As template, one can use hypothalamus cDNA (Clontech). The PCR product was ligated into the mammalian expression vector pcDNA3.1/V5-His-TOPO (Invitrogen) according to the manufacturers recommendations. The resulting insert was subsequently sequence-verified on both strands using ABI DNA sequencing methodology as per manufacturers protocol.

24-72 hours after transient transfection using standard procedures (e.g. using Lipofectamine (Invitrogen), following the manufacturer's recommendations), transfected CHO cells are harvested by scraping, resuspended in 20 ml of ice-cold assay buffer (50 mM Tris-HCl pH 7.4), homogenised, and the resulting suspension is centrifuged at 20,000 g, 4° C. for 30 minutes. The supernatant is decanted, the pellet resuspended in 3 ml of assay buffer and re-homogenised (50 mM Tris-HCl pH7.4). The protein concentration is determined via Bradford's assay (Biorad), according to the manufacturer's recommendations.

Aliquots of this membrane preparation containing 200 μg protein are then incubated with the test compounds and radiolabelled Kiss-1 or Kiss-1 fragment (e.g. metastin (40-54) labelled with ¹²⁵I-Na using lactoperoxidase, purified to a single peak; see Ohtaki et al (2001) Nature 411, 613-617), for about 2 hrs at room temperature or at 30° C. To terminate incubations, samples are rapidly filtered using the Brandell cell harvester onto Wallac Filtermats (Perkin Elmer) (which have been previously soaked (for 1 h) in a 0.3% (v/v) solution of PEI (polyethylenimine; Sigma) in assay buffer to reduce Filtermat binding). Immediately, the Filtermat/wells are washed four times in rapid succession with 2 ml of assay buffer per well. Filtermats are dried using a microwave oven, and Meltilex scintillant (Perkin Elmer) is melted onto the Filtermats using the Wallac Meltilex heat sealer. The bound radioactivity on the Filtermats is determined using the Wallac betaplate scintillation counter.

The specific binding is defined as the difference between total radioactivity bound minus the radioactivity measured in the presence of an excess of unlabelled ligand. Mock-transfected cells are also measured to assess whether the host cells express receptors for the ligands used endogenously.

To determine selectivity, the antagonists can be screened against the receptors that are closely related to the Kiss-1 receptor using appropriate radioligands and binding conditions for each receptor.

### Example 2

### Identification of Kiss-1 Antagonists Measuring the Inhibition of Agonist-induced Rise in Intracellular Calcium by Test Compounds

CHO cells transiently transfected to express Kiss-1 receptor were prepared as described above. Approximately 24 hrs post-transfection, the cells were detached from the flask using Trypsin/EDTA solution (LTI) and seeded into a black sided, Poly-D-lysine-treated, 96-well plate (Becton Dickinson) at 5×10⁴ cells/well density. The plates were left overnight to allow the cells to adhere to the bottom of the wells. The medium was removed from the cells and replaced with 100 μl warm (37° C.) dye loading solution (56 μg Fluo3 (Molecular Probes) in 20 μl DMSO+20% pluronic acid in DMSO, added to 11 ml Dulbecco's Modified Eagles Medium containing 1× Probenecid (100× Probenecid-0.71 g Probenecid was dissolved in 5 ml 1M NaOH and 5 ml Dulbeccos' Phosphate Buffered Saline (PBS), per plate; Probenecid (Molecular Probes) inhibits activity of the anion transport protein, thus improving dye loading). The plates were then incubated for 1 hr at 37° C. Plates were subsequently washed with 250 μl of wash buffer per well (5 ml 100× Probenecid stock+495 ml PBS, pH 7.4) 4 times. The plates were returned to the 37° C./5% CO₂ incubator for 30 mins prior to processing within the FLIPR® instrument. The FLIPR® processing involves reading the fluorescence for all samples for 2 minutes; during this time the fluorescence baseline is determined for 10 seconds. The desired amount of compounds (i.e. potential agonists) was then automatically transferred to the wells and the fluorescence was continuously monitored for the remainder of the time. All compounds were diluted in wash buffer.

Compounds capable of acting as agonists for the receptor were identified by them causing a transient rise in fluorescence, and therefore in intracellular calcium, in the cells. Compounds capable of acting as antagonist were identified by pre-incubating the test compounds with the cells, prior to addition of a Kiss-1 agonist (e.g. Kiss (45-54), whereby antagonists were identified as inhibiting the transient rise in fluorescence seen by the agonist in the absence of test compound.

### Example 3

### Isolated Rat Aorta Assay

Segments of endothelium intact rat aorta were trim cleaned of fat and surrounding connective tissues and aortic rings strung up in 5 ml organ baths and perfused with oxygenated modified Kreb's solution (118 mM NaCl, 4.7 mM KCl, 25 mM NaHCO₃, 11.1 mM glucose, 1.18 mM KH₂PO₄, 1.18 mM MgSO₄ and 2mM CaCl₂). Following an equilibration period of approximately 60 min, tissues were challenged with 1 μM phenylephrine (PE). Following a stable response tissues were washed and the above dose of PE was repeated. Once the tissues reached a maximum response 1 μM acetylcholine was administered to determine the percentage of endothelium dependent relaxation. Tissues were washed and cumulative concentration response curves were constructed to Kiss-1 and urotensin-II for a comparison. FIG. 1 shows the dose-response curve for Kiss-1 (45-54). The potential for Kiss-1 mediated vasorelaxation was assessed in aortic rings pre-constricted with 300 nM noradrenaline along with appropriate control tissues with no drug. Drug potency (EC₅₀) and efficacy (Eₘₐₓ) was determined.

Rat aortic rings are set up as described before for measuring Kiss-1 potency and efficacy in a 5 ml organ bath set-up. The potency of antagonist can be determined by constructing a dose response curve to Kiss-1 in the presence of a single concentration of Kiss-1 antagonist or vehicle. The pA₂ values for Kiss-1 antagonists against Kiss-1 can be derived across tissues on a per experiment basis from Schild plots (Arunlakshana, O. and Schild, H. O. (1959) Br. J. Pharmacol. 14, 48-58).

### Example 4

### Animal Model to Assess the Effect of Kiss-1 Antagonists in the Treatment of Hypertension

The efficacy of the combinations of the invention may be determined in the spontaneously hypertensive rat, which is a widely used model of human hypertension. Animals are instrumented with Doppler flow probes for the measurement of mesenteric, hindquarters and renal blood flow, aortic blood pressure and heart rate according to published methods (Gardiner, S M et al. (2001); Br. J. Pharmacol. 132(8), 1625-1629). Baseline haemodynamic parameters are recorded. Animals (n=3/group) are then treated with a Kiss-1 antagonist by continuous infusion over 80 hours. A control group of animals receives saline. Changes in haemodynamic parameters are monitored during the study period.

1. A method of treating hypertension comprising administering to a mammal in need of such treatment a therapeutically effective amount of a compound which is a Kiss-1 receptor antagonist.

2. The method of claim 1 wherein said Kiss-1 receptor antagonist has an IC₅₀ for the Kiss-1 receptor less than 100 nM.

3. The method of claim 1 wherein said Kiss-1 receptor antagonist is selective for the Kiss-1 receptor.

4. A method of screening for compounds useful for the treatment of hypertension, comprising screening compounds for antagonist activity against Kiss-1 receptor, and selecting compounds with an IC₅₀ of less than 100 nM.

5. A process for providing a medicament for the treatment of hypertension, comprising the following steps:
(a) testing compounds in a ligand binding assay against the Kiss-1 receptor;
(b) selecting a compound with an IC₅₀ of less than 100 nM;
(c) formulating a compound with the same structure as that selected in step (b), or a pharmaceutically acceptable salt thereof, with a pharmaceutically acceptable carrier or excipient.

6. The process of claim 5, additionally comprising the following steps:
(a) packaging the formulation of step (c);
(b) making the package of step (d) available to a patient suffering from hypertension.

7. A process for providing a medicament for the treatment of hypertension, comprising the following steps:
(a) testing compounds in an assay, measuring the inhibition of the agonist-stimulated second messenger response of Kiss-1 receptors;
(b) selecting a compound with an IC₅₀ of less than 100 nM;
(c) formulating a compound with the same structure as that selected in step (b), or a pharmaceutically acceptable salt thereof, with a pharmaceutically acceptable carrier or excipient.

8. The process of claim 7, additionally comprising the following steps:
(d) packaging the formulation of step (c);
(e) making the package of step (d) available to a patient suffering from hypertension.

9. A process for preparing a medicament for the treatment of hypertension, comprising the steps of (a) testing compounds in a ligand binding assay against Kiss-1 receptor or testing compounds in an assay, measuring the inhibition of the agonist-stimulated second messenger response of Kiss-1 receptors, (b) identifying one or more compounds capable of antagonising the Kiss-1 receptor with an IC₅₀ of less than 100 nM; and (c) preparing a quantity of those one or more identified compounds.

10. A method of preparing a composition for treating hypertension which comprises:
(a) identifying a compound which specifically binds to the Kiss-1 receptor by a method which comprises contacting cells expressing Kiss-1 receptor or membranes prepared from such cells with a radiolabelled Kiss-1 receptor ligand in the presence or absence of a test compound, measuring the radioactivity bound to the cells or membranes, comparing the radioactivity bound to the cells or membranes in the presence and absence of test compound, whereby a compound which causes a reduction in the radioactivity bound is a compound specifically binding to Kiss-1 receptor; and
(b) admixing said compound with a carrier.

11. A method of preparing a composition for treating hypertension which comprises:
(a) identifying a compound which specifically binds to and inhibits the activation of a Kiss-1 receptor by a method which comprises separately contacting cells expressing Kiss-1 receptor on their surface and producing a second messenger response in response to Kiss-1 or a Kiss-1 agonist, or a membrane preparation of such cells, with both the compound and an agonist of the Kiss-1 receptor, and with only the agonist, under conditions suitable for activation of the Kiss-1 receptor, and measuring the second messenger response in the presence of only the agonist for the Kiss-1 receptor and in the presence of the agonist and the compound, a smaller change in the second messenger response in the presence of both agonist and compound than in the presence of the agonist only indicating that the compound inhibits the activation of the Kiss-1 receptor; and
(b) admixing said compound with a carrier.

12. A method of treating a blood pressure anomaly comprising administering to a mammal in need of such treatment a therapeutically effective amount of a compound comprising a Kiss-1 receptor antagonist wherein said blood pressure anomaly is hypertension or a Kiss-1 receptor agonist wherein said blood pressure anomaly is hypotension.


SEQUENCE LISTING (6 sequences)

SEQ ID NO: 1 - DNA, 1197 bp, homo sapiens
atgcacaccgtggctacgtccggacccaacgcgtcctggggggcaccggccaacgcctccggctgcccgggctgtggcgccaacgcctcggacggcccagtcccttcgccgcgggccgtggacgcctggctcgtgccgctcttcttcgcggcgctgatgctgctgggcctggtggggaactcgctggtcatctacgtcatctgccgccacaagccgatgcggaccgtgaccaacttctacatcgccaacctggcggccacggacgtgaccttcctcctgtgctgtgtccccttcacggccctgctgtacccgctgcccggctgggtgctgggcgacttcatgtgcaagttcgtcaactacatccagcaggtctcggtgcaggccacgtgtgccactctgaccgccatgagtgtggaccgctggtacgtgacggtgttcccgttgcgcgccctgcaccgccgcacgccccgcctggcgctggctgtcagcctcagcatctgggtaggctctgcggcggtgtctgcgccggtgctcgccctgcaccgcctgtcacccgggccgcgcgcctactgcagtgaggccttccccagccgcgccctggagcgcgccttcgcactgtacaacctgctggcgctgtacctgctgccgctgctcgccacctgcgcctgctatgcggccatgctgcgccacctgggccgggtcgccgtgcgccccgcgcccgccgatagcgccctgcaggggcaggtgctggcagagcgcgcaggcgccgtgcgggccaaggtctcgcggctggtggcggccgtggtcctgctcttcgccgcctgctggggccccatccagctgttcctggtgctgcaggcactgggccccgcgggctcctggcacccacgcagctacgccgcctacgcgcttaagacctgggctcactgcatgtcctacagcaactccgcgctgaacccgctgctctacgccttcctgggctcgcacttccgacaggccttccgccgcgtctgcccctgcgcgccgcgccgcccccgccgcccccgccggcccggaccctcggaccccgcagccccacacgcggagctgcaccgcctggggtcccacccggcccccgccagggcgcagaagccagggagcagtgggctggccgcgcgcgggctgtgcgtcctgggggaggacaacgcccctctctga

SEQ ID NO: 2 - DNA, 1188 bp, Rattus norvegicus
atggccgcagaggcgacgttgggtccgaacgtgagctggtgggctccgtccaacgcttcgggatgcccgggctgcggtgtcaatgcctcggatggcccaggctccgcgccaaggcccctggatgcctggctggtgcccctgtttttcgctgccctaatgttgctggggctagtcgggaactcactggtcatcttcgttatctgccgccacaagcacatgcagaccgtcaccaatttctacatcgctaacctggcggccacagatgtcactttccttctgtgctgcgtacccttcaccgcgctcctctatccgctgcccacctgggtgctgggagacttcatgtgcaaattcgtcaactacatccagcaggtctcggtgcaagccacatgtgccactttgacagccatgagtgtggaccgctggtacgtgactgtgttcccgctgcgtgcacttcaccgccgcactccgcgcctggccctgactgtcagccttagcatctgggtgggttccgcagctgtttccgccccggtgctggctctgcaccgcctgtcgcccgggcctcacacctactgcagtgaggcgtttcccagccgtgccctggagcgcgctttcgcgctctacaacctgctggccctatacctgctgccgctgctcgccacctgcgcctgctacggtgccatgctgcgccacctgggccgcgccgctgtacgccccgcacccactgatggcgccctgcaggggcagctgctagcacagcgcgctggagcagtgcgcaccaaggtctcccggctggtggccgctgtcgtcctgctcttcgccgcctgctggggcccgatccagctgttcctggtgcttcaagccctgccgctcgggggcctggcaccctcgaagctatgcgcctacgcgctcaagatctgggctcactgcatgtcctacagcaattctgcgctcaacccgctgctctatgccttcctgggttcccacttcagacaggccttctgccgcgtgtgcccctgcggcccgcaacgccagcgtcggccccacgcgtcagcgcactcggaccgagccgcaccccatagtgtgccgcacagccgggctgcgcaccctgtccgggtcaggacccccgagcctgggaaccctgtggtgcactcgccctctgttcaggatgaacacactgccccactctga

SEQ ID NO: 3 - DNA, 1191 bp, Mus musculus
atggccaccgaggcgacattggctcccaatgtgacctggtgggctccgtccaacgcttcaggatgcccaggctgcggtgtcaacgcctcggatgacccaggctctgcgccaaggcccctggatgcctggctggttcccctgtttttcgctacactcatgttgcttgggctggtcggaaactcattggtcatctacgttatctgccgccacaagcacatgcagacagttaccaacttctacatcgctaacctggctgccacagacgtcactttcctactgtgctgcgtgcccttcaccgcactcctctacccgctgcccgcctgggtgctgggagacttcatgtgcaaattcgtcaactacatccagcaggtctcggtgcaagccacatgtgccactctgacggccatgagtgtggaccgctggtatgtgactgtgttcccgctgcgtgcacttcaccgccgcactccgcgcctggccctggctgtcagcctcagcatctgggtggggtcagcagctgtgtccgccccggtgctggccctgcaccgcctgtcgccagggcctcgcacctactgcagcgaggcgtttcccagccgcgccctggagcgcgccttcgcgctctacaacctgctggctctatatctgctgccgctgctcgccacctgcgcctgctacggcgccatgctgcgccacctgggccgtgcggctgtacgccccgcacccactgacggcgccctgcagggacagctgctagcacagcgcgccggagcagtgcgcaccaaggtctcccggctggtggccgctgtcgtcctgctcttcgccgcctgctggggcccgatccagctgttcctggtgcttcaagccctgggcccctcgggggcctggcaccctcgaagctatgccgcctacgcggtcaagatctgggctcactgcatgtcctacagcaactcggcgctcaatccgctgctctatgccttcctgggttcacacttcagacaggccttctgccgcgtgtgcccctgctgccggcaacgccagcgccggccccacacgtcagcgcactcggaccgagctgcaactcacactgtgccgcacagccgtgctgcgcaccctgtgcggatcaggagcccggagcctgggaaccctgtggtgcgctcgccctgcgctcagagtgaacgcactgcctcactctga

SEQ ID NO: 4 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELHRLGSHPAPARAQKPGSSGLAARGLCVLGEDNAPL

SEQ ID NO: 5 - protein, 395 aa, Rattus norvegicus
MAAEATLGPNVSWWAPSNASGCPGCGVNASDGPGSAPRPLDAWLVPLFFAALMLLGLVGNSLVIFVICRHKHMQTVTNFYIANLAATDVTFLLCCVPFTALLYPLPTWVLGDFMCKFVNYIQQVSVQATCATLTAMSVDRWYVTVFPLRALHRRTPRLALTVSLSIWVGSAAVSAPVLALHRLSPGPHTYCSEAFPSRALERAFALYNLLALYLLPLLATCACYGAMLRHLGRAAVRPAPTDGALQGQLLAQRAGAVRTKVSRLVAAVVLLFAACWGPIQLFLVLQALPLGGLAPSKLCAYALKIWAHCMSYSNSALNPLLYAFLGSHFRQAFCRVCPCGPQRQRRPHASAHSDRAAPHSVPHSRAAHPVRVRTPEPGNPVVHSPSVQDEHTAPL

SEQ ID NO: 6 - protein, 396 aa, Mus musculus
MATEATLAPNVTWWAPSNASGCPGCGVNASDDPGSAPRPLDAWLVPLFFATLMLLGLVGNSLVIYVICRHKHMQTVTNFYIANLAATDVTFLLCCVPFTALLYPLPAWVLGDFMCKFVNYIQQVSVQATCATLTAMSVDRWYVTVFPLRALHRRTPRLALAVSLSIWVGSAAVSAPVLALHRLSPGPRTYCSEAFPSRALERAFALYNLLALYLLPLLATCACYGAMLRHLGRAAVRPAPTDGALQGQLLAQRAGAVRTKVSRLVAAVVLLFAACWGPIQLFLVLQALGPSGAWHPRSYAAYAVKIWAHCMSYSNSALNPLLYAFLGSHFRQAFCRVCPCCRQRQRRPHTSAHSDRAATHTVPHSRAAHPVRIRSPEPGNPVVRSPCAQSERTASL